(19) United States
(12) Patent Application Publication
(10) Pub. No.: US 2007/0142417 A1
(43) Pub. Date: Jun. 21, 2007

(54) Substantially Pure 2-{[2-(2-Methylamino-Pyrimidin-4-YL)-1H-Indole-5-Carbonyl]-Amino}-3-Phenylpyridin-2-YL-Amino)-Propionic Acid as an IkB Kinase Inhibitor

(75) Inventors: El-Bdaoui HADDAD, Basking Ridge, NJ (US); Olaf Ritzeler, Bad Soden (DE); David J. Aldous, Gillette, NJ (US); Paul Joseph Cox, Flemington, NJ (US)

Correspondence Address: ROSS J. OEHLER;SANOFI-AVENTIS U.S. LLC, 1041 ROUTE 202-206, MAIL CODE: D303A, BRIDGEWATER, NJ, 08807, US

(73) Assignee: AVENTIS PHARMACEUTICALS INC, Bridgewater, NJ (US)

(21) Appl. No.: 11/559,159

(22) Filed: Nov. 13, 2006

Related U.S. Application Data
(63) Continuation of application No. PCT/EP05/16381, filed on May 11, 2005.

(51) Int. Cl. A61K 31/506; C07D 403/14

(52) U.S. Cl. 514/275; 544/331

(57) ABSTRACT

The present invention is directed to the substantially pure compound of formula (A), 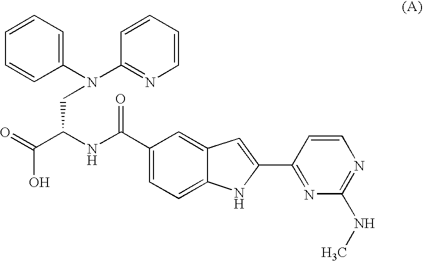 or pharmaceutically acceptable salt, or solvate of said compound; to a pharmaceutical composition comprising a pharmaceutically effective amount of the compound of formula (A), and a pharmaceutically acceptable carrier; and the use of a compound of formula (A) having activity as an inhibitor, preferably a selective inhibitor, of IκB (IKK), particularly IKK-2, and methods related thereto.

### CROSS-REFERENCE TO RELATED APPLICATIONS

This application is a continuation of international application No. PCT/US2005/016381, filed May 11, 2005, which claims the benefit of priority from U.S. provisional application No. 60/570,146, filed on May 12, 2004.

### FIELD OF THE INVENTION

This invention is directed to an indole derivative, its preparation, a pharmaceutical composition comprising the compound, its use, and intermediates thereof.

### BACKGROUND OF THE INVENTION

NF-κB is a heterodimeric transcription factor that regulates the expression of multiple inflammatory genes. The expression of more than 70 known proteins is transcriptionally regulated by the binding of NF-κB to specific sequence elements in the promoter region of these genes (Baeuerle and Baichwal, Advances in Immunology 65:111-137, 1997). NF-κB has been implicated in many pathophysiologic processes including angiogenesis (Koch et al., Nature 376:517- 519, 1995), atherosclerosis (Brand et al., J Clin Inv. 97:1715-1722, 1996), endotoxic shock and sepsis (Bohrer et al., J. Clin. Inv. 100:972-985, 1997), inflammatory bowel disease (Panes et al., Am J Physiol. 269:H1955-H1964, 1995), ischemia/reperfusion injury (Zwacka et al., Nature Medicine 4:698 704, 1998), and allergic lung inflammation (Gosset et al., Int Arch Allergy Immunol. 106:69-77, 1995). Thus the inhibition of NF-κB by targeting regulatory proteins in the NF-κB activation pathway represents an attractive strategy for generating anti-inflammatory therapeutics due to NF-κB's central role in inflammatory conditions.

the IκB kinases (IKKs) are key regulatory signaling molecules that coordinate the activation of NF-κB. Two IKKs, IKK-1 (IKKα)and IKK-2 (IKK-β), are structurally unique kinases containing an N-terminal kinase domain with a dual serine activation loop, a leucine zipper domain, and a C-terminal-helix-loop-helix domain and serine cluster. IKK enzymes show relatively low sequence homologies with other kinases, and early profiles with known kinase inhibitors have not identified compounds with with other kinases, and early profiles with known kinase inhibitors have not identified compounds with striking potency. Kinetic analysis shows that IKK-2 binds to and phosphorylates IκBα, and IκBεwith high and relatively equal affinities (Heilker et al. 1999). Recombinant IKK-2 phosphoylates IκBα peptide 26-42 with near equal affinity to full length IκBα, however the native IKK enzyme complex phosphorylates full length IκBα 25,000 fold more efficiently, suggesting important regulatory sequences in the C-terminal region of IκBα, or additional regulatory proteins in the IKK enzyme complex that accelerate the rate of catalysis (Burke et al., Journal of Biological Chemistry 274;36146-36152, 1999). Phosphorylation of IκBα occurs via a random sequential kinetic mechanism, meaning either ATP or IκBα may bind first to IKK-2, that both must be bound before phosphorylation of IκBα can take place (Peet and Li, Journal of Biological Chemistry 274;32655-32661, 1999). IKK-2 binds ATP with uniquely high affinity (Ki=130 nM) compared to other serine-threonine kinases such as p38 and JNK perhaps indicating a unique ATP binding pocket that reflects the relatively poor activity to many broad specificity kinase inhibitors when tested against IKK-2. To date, no crystal structure of IKK-2 has been reported. However homology modeling has identified 3 structural domains including an N-terminal kinase domain with an activation loop, a leucine zipper domain that likely mediates the formation of IKK-1 and IKK-2 homo/heterodimers, and a C-terminal helix-loop-helix with serine rich tail. Activation of IKK-2 is dependent upon phosphorylation of serine 177 and 181 in the activation or T loop. Alanine mutations abolish activity, while glutamate mutations result in a constitutively active enzyme (Mercurio et al. Science 278:860-866, 1997; Delhase et al., Science 248:30 313, 1999).

IKK-1 and IKK-2 occur both as heterodimers and IKK-2 homodimers, and are associated with a 700-900 kDa cytoplasmic enzyme complex called the “IKK Signalsome” (Mercurio et al., Science 278:860-866, 1997). another component, IKKAP-1 or NEMO/IKKγ has no apparent catalytic function but will associate directly with IKK-2 and is necessary for full activation of NF-κB (Mercurio et al., Mol Cell Biol 19:1526-1538, 1999). Many immune and inflammatory mediators including TNFα, lipopolysaccharide (LPS), IL-1β, CD3/CD28 (antigen presentation), CD40L, FasL, viral infection, and oxidative stress have been shown to lead to NF-κB activation. Although the receptor complexes that transduce these diverse stimuli appear very different in their protein components, it is understood that each of these stimulation events leads to activation of the IKKs and NF-κB.

the IKK complex appears to be the central integrator of diverse inflammatory signals leading to the phosphorylation of IκB. IKKs are activated at dual serine residues by upstream kinases including NF-κB inducing kinase, NIK (Malinin et al., Nature 385;540-544, 1997), and MEKK-1 (Yujiri et al., Science 282;1911-1914, 1998). the differential activities of NIK and MEKK-1 remain unclear although initial data indicates these kinases may preferentially activate IKK-1 and IKK-2, respectively.

Activated IKK phosphorylates a cytoplasmic inhibitor protein, IκB that binds NF—κB, thereby masking a nuclear localization signal present in Rel proteins (Cramer et al., Structure 7:R1-R6, 1999). IKK phosphorylation of IκB on serines 32 and 36 forms a structural motif recognized by the E3 ligase, βTRcP (Yaron et al., Nature 396:590-594, 1998). Docking of βTRcP results in the formation of a ligase complex which polyubiquitinates IκB thus targeting it for degradation by the 26S proteosome. Free NF—κB is then identified by nuclear transport proteins, which translocate it to the nucleus where it can associate with sequence specific regulatory elements on gene promoters.

Although both kinases can phosphorylate IκB in vitro, early studies using genetic mutants indicated that IKK-2, but not IKK-1, was essential for activation of NF—κB by proinflammatory stimuli such as IL-1β and TNFα. Furthermore, only catalytically inactive mutants of IKK-2 blocked the expression of NF-κB regulated genes such as monocyte chemotactic protein (MCP-1) and intercellular adhesion molecule (ICAM-1) (Mercurio et al, Science 278:860-866, 1997). studies of knockout animals for IKK-1 and IKK-2 substantiate these initial findings (Hu et al., Science 284:316-320, 1999; Li et al., Genes & Development 13:1322-1328, 1999; Li et al., Science 284;321-324, 1999; Takeda et al., Science 84:313-316, 1999; Tanaka et al., Immunity 10:421-429, 1999). IKK-1^{-/-} animals were born alive but died within hours. Pups showed abnormalities of the skin due to defective proliferation and differentiation, but showed no gross deficiency in cytokine induced activation of NF-κB. In contrast, IKK-2^{-/-} embryos died at day 14-16 of pregnancy from liver degeneration and apoptosis that bore a striking resemblance to that observed in Rel A knock-out animals (Beg et al., Nature 376;167-170, 1995). Furthermore, embryonic fibroblasts from IKK-2^{-/-} animals exhibited markedly reduced NF-κB activation following cytokine stimulation, while IKK-1^{-/-} did not.

Accordingly, cell and animal experiments indicate that IKK-2 is a central regulator of the pro-inflammatory role of NF-κB, wherein the IKK-2 is activated in response to immune and inflammatory stimuli and signaling pathways. Many of those immune and inflammatory mediators, including IL-1β, LPS, TNFα, CD3/CD28 (antigen presentation), CD40L, FasL, viral infection, and oxidative stress, play an important role in respiratory diseases. Furthermore, the ubiquitous expression of NF-κB, along with its response to multiple stimuli means that almost all cell types present in the lung are potential target for anti-NF-κB/IKK-2 therapy. This includes alveolar epithelium, mast cells, fibroblasts, vascular endothelium, and infiltrating leukocytes, including neutrophils, macrophages, lympophocytes, eosinophils and basophils. By inhibiting the expression of genes such as cyclooxygenase-2 and 12-lipoxygenase (synthesis of inflammatory mediators), TAP-1 peptide transporter (antigen processing), MHC class I H-2K and class II invariant chains (antigen presentation), E-selectin and vascular cell adhesion molecule (leukocyte recruitment), interleukins-1, 2, 6, 8 (cytokines), RANTES, eotaxin, GM-CSF (chemokines), and superoxide dismutase and NADPH quinone oxidoreductase (reactive oxygen species) inhibitors of IKK-2 are believed to display broad anti-inflammatory activity.

Patent application WO 94/12478, the content of which is incorporated herein by reference, describes, inter alia, indole derivatives that inhibit blood platelet aggregation. Patent applications WO 01/00610 and WO 01/30773, the content of each of which is incorporated herein by reference, describe indole derivatives and benzimidazole derivatives, which are able to modulate NF-κB. As described above, NF-κB is a heterodimeric transcription factor that is able to activate a large number of genes that encode, inter alia, proinflammatory cytokines such as IL-1, IL2, TNFα or IL-6. NF-κB is present in the cytosol of cells, where it is complexed with its naturally occurring inhibitor IκB. Stimulation of the cells, for example by cytokines, leads to the IκB being phosphorylated and subsequently broken down proteolytically. This proteolytic breakdown leads to the activation of NF-κB, which then migrates into the nucleus of the cell, where it activates a large number of proinflammatory genes.

In diseases such as rheumotoid arthritis, osteoarthritis, asthma, chronic obstructive pulmonary disorder (COPD), rhinitis, multiple sclerosis, cardiac infarction, Alzheimer's diseases, diabetes Type II, inflammatory bowel disease or artherosclerosis, NF-κB is activated beyond its normal extent. The inhibition of NF-κB is also described as being useful for treating cancer on its own or in addition to cytostatic therapy. Inhibition of the NF-κB-activating signal chain at various points or by interfering directly with the transcription of the gene by glucocorticoids, salicylates or gold salts, has been shown as being useful for treating rheumatism.

The first step in the avovementioned signal cascade is the breakdown of IκB. This phosphorylation is regulated by the specific IκB kinase. Thus far, inhibitors of IκB kinase are known to frequently suffer from the disadvantage of being non-specific for inhibiting only one class of kinases. For example, most inhibitors of IκB kinase inhibit several different kinases at the same time because the structure of the catalytic domains of these kinases are similar. Consequently, the inhibitors act, in an undesirable manner on many enzymes, including those that possess the vital function.

Chronic obstructive pulmonary disease (COPD) is a debilitating inflammatory disease of the lungs characterized by the progressive development of airflow limitation that is not fully reversible (Pauwels et al., 2000). The airflow limitation is associated with an abnormal inflammatory response of the lungs to noxious particles or gases, primarily caused by cigarette smoking. Although COPD affects the lungs, it also produces significant systemic consequences. The term COPD encompasses chronic obstructive bronchitis, with obstruction of small airways, and emphysema, with enlargement of air spaces and destruction of lung parenchyma, loss of lung elasticity, and closure of small airways. Chronic bronchitis, by contrast, is defined by the presence of a productive cough (due to hypersecretion of mucus) of more than three months' duration for more than two successive years. There is some epidemiologic evidence that mucus hypersecretion is accompanied by airflow obstruction, perhaps as a result of obstruction of peripheral airways. Most patients with COPD have all three pathologic conditions (chronic obstructive bronchitis, emphysema, and mucus plugging), but the relative extent of emphysema and obstructive bronchitis within individual patients can vary, Vestbo et al., 1996; Barnes, 2004a, Barnes, 2004b; Hogg, 2004.

In industrialized countries, cigarette smoking accounts for most cases of COPD, but in developing countries other environmental pollutants (particularly with sulfur dioxide and particulates) and certain occupational chemicals (such as cadmium), are important causes. Passive smoking is also a risk factor.

COPD patients are predisposed to exacerbations, that is, an acute worsening of their respiratory symptoms. An exacerbation of COPD is an event in the natural course of the disease characterized by a change in the patient's baseline dyspnea, cough and/or sputum beyond day-to-day variability sufficient to warrant a change in management (Rodriguez-Roisin, 2000; Burge and Wedzicha, 2003).

Tracheobronchial infections are believed to be a common cause of exacerbation in COPD, although controversy exists regarding the nature of the infectious agent as well as its exact role (Wedzicha, 2002; White et al., 2003). In addition, exacerbations of COPD are clearly associated with the levels of respirable particles and environmental air pollutants, and these have been linked to hospital admission rates (Rennard and Farmer, 2004).

The frequency of exacerbations is linked to disease severity in COPD. Exacerbations, may adversely affect the natural history of these disorders, perhaps by contributing to increased rates of lung function decline, systemic effects, and premature mortality. Unfortunately, to date, there is now widely accepted definition of what constitutes an exacerbation of COPD (Rodriguez-Roisin, 2000). The intensity and duration of increased symptoms required to qualify as an “exacerbation” are difficult to define. Indeed, several definitions co-exist, and many clinical trials employ substantially different criteria or describe poorly the definition(s) used to diagnose exacerbation. The most widely quoted clinical criteria used in the characterization of acute exacerbation of COPD are those described by Anthonisen et al., (1987). In that study exacerbations were divided into three groups: type 1 exacerbations were characterized by increased breathlessness, increased sputum volume, and new or increased sputum purulence; type 2 included any two of these symptoms; and type 3 consisted of any one of the symptoms together with at least one additional feature, including sore throat or nasal discharge within the last five days; unexplained fever; increased wheeze; increased cough; or a 20% increase in respiratory or heart rate compared with baseline. These criteria have been used as a benchmark ever since, and all proposed etiologies of exacerbation need to establish their relationship to these key features.

The inhibition of NF-κB is also described as being useful for treating hypoproliferative diseases, e.g., solid tumor and leukemias, on its own or in addition to cytostatic therapy. Inhibition of the NF-κB-activating signal chain at various points or by interfering directly with the transcription of the gene by glucocorticoids, salicylates or gold salts, has been shown as being useful for treating rheumatism.

Patent application WO 01/30774 discloses indole derivatives and U.S. application Ser. No. 10/642,970, discloses indole and benzimidazole derivatives which are able to modulate NF-κB and which exhibit a strong inhibitory effect on IκB kinase. Particulary, U.S. application Ser. No. 10/642,970 discloses indole and benzimidazole derivatives of formula (I), their preparation, 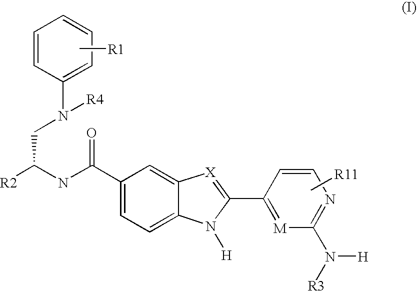
pharmaceutical compositions containing these compounds, and methods for the prophylaxis and therapy of a disease associated with an increased activity of IκB kinase comprising administering such compounds. Furthermore, U.S. application Ser. No. 10/642,970 discloses the following compounds of formulae (B), (C), and (D):

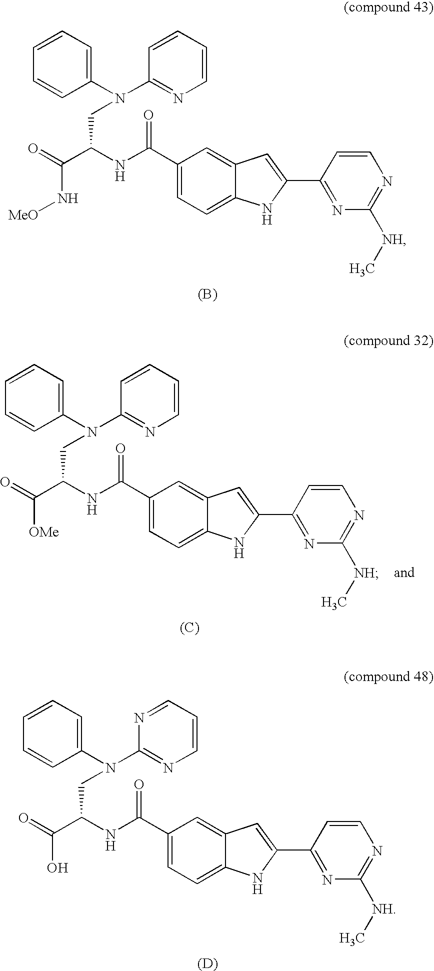

However, U.S. application Ser. No. 10/642,970 does not specifically disclose the compound of Formula (I) wherein M is N; R1 is hydrogen, R2 is carboxyl (—COOH), R3 is methyl R4 is pyridin-2-yl, R11 is hydrogen, and X is CH.

In view of the aforesaid, there is a need for an inhibitor of IκB kinase that operates through the selective inhibition of IKK, particulary an IKK-2 inhibitor. Also desired would be to have such an inhibitor that exhibits a localized activity as opposed to a systemic activity. Such an inhibitor should have a utility in treating a patient suffering from or subject to IKK-2 mediated pathological (diseases) conditions, e.g., asthma, or a chronic obstructive pulmonary disorder (COPD), that could be ameliorated by the targeted administering of then inhibitor.

### SUMMARY OF THE INVENTION

The present invention is directed to a compound having activity as an inhibitor, preferably a selective inhibitor, of IκB (IKK), particularly IKK-2, and to a composition and methods related thereto.

In particular, the present invention is directed to the substantially pure compound of formula (A): 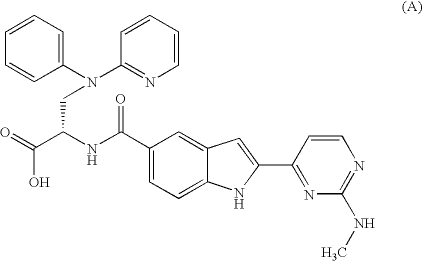
or a pharmaceutically acceptable salt, or solvate of said compound.

Furthermore, the present invention is directed to a pharmaceutical composition comprising a pharmaceutically effective amount of the compound of formula (A), and a pharmaceutically acceptable carrier.

Furthermore, the present invention is directed to the use of a compound of formula (A) as an inhibitor of IκB kinase.

### BRIEF DESCRIPTION OF THE DRAWINGS

FIG. 1 shows a picture of mouse imaging studies in the NF-κB-Luciferase Mouse Model wherein animals are treated with the Vehicle/PBS solution (negative control animals) and wherein animals have IL-1β-induced NF-κB activation without any compound present [Vehicle/IL-1β] or with increasing doses (0.3 mpk, 3 mpk, and 10 mpk) of Compound (A) or Compound (B).

FIG. 2 shows a graph of mouse imaging studies in the NF-κB-Luciferase Reporter Mouse Model showing bioluminescence levels for animals treated with the Vehicle/PBS solution (negative control animals) and wherein animals have IL-1β-induced NF-κB activation without any compound present [Vehicle/IL-1β] or with increasing doses (0.3 mpk, 1 mpk, 3 mpk, and 10 mpk) of Compound (A) or Compound (B).

FIG. 3, on the left, shows a graph of Compound (A) levels after i.t. instillation of 0.3 mg/kg of Compound (A) in lung and plasma tissue; and on the right, shows a graph of Compound (A) and Compound (B) levels after i.t. instillation of 0.3 mg/kg of Compound (B) in lung and plasma tissue.

FIG. 4, on the left, shows a graph of the lung exposure of Compound (A) after administering increasing doses (0.01 mpk, 0.03 mpk, 0.10 mpk, and 0.030 mpk) of Compound (A); and on the right, shows a graph of the plasma exposure of Compound (A) after administering increasing doses (0.01 mpk, 0.03 mpk, 0.10 mpk, and 0.30 mpk) of Compound (A).

FIG. 5, on the left, shows a graph of the lung exposure of Compounds (A) and (B) after administering increasing doses ().01 mpk, 0.03 mpk, 0.10 mpk, and 0.30 mpk) of Compound (B); and on the right, shows a graph of the plasma exposure of Compounds (A) and (B) after administering increasing doses (0.01 mpk, 0.03 mpk, 0.10 mpk, and 0.30 mpk) of Compound (B).

### DETAILED DESCRIPTION

List of Abbreviations

As used above, and throughout the description of the invention, the following abbreviations, unless otherwise indicated, shall be understood to have the following meanings:

| | |
|---|---|
| Boc₂O | di-tert-butyl dicarbonate |
| DIEA | N,N-diisopropylethylamine |
| DMAP | 4-dimethylaminopyridine |
| DMF | dimethylformamide |
| DMSO | dimethylsulfoxide |
| ESI-MS | electrospray ionization mass spectrometry |
| FAB-MS | fast-atom bombardment mass spectrometry |
| HATU | O-(7-azabenzotriazol-1-yl)-N,N,N′,N′- |
| | tetramethyluronium |
| | hexaflurophosphate |
| HPLC | high performance liquid chromatography |
| PBS | phosphate-buffered saline |
| i.n. | intranasally |
| PO | oral |
| i.p. | intraperitoneal |
| i.t. | intra-tracheally |
| Microcystin-LR | liver toxin produced by certain cyanobacteria of the |
| | genera Anabaena and Oscillatoria |
| mbr | millibar |
| mpk | mg/kg |
| HEPES | 4-(2-hydroxyethyl)-1-piperazineethanesulfonic acid |
| DTT | dithiothreitol |
| ATP | adenosine triphosphate |
| streptavidin-HRP | Streptavidin-Horseradish Peroxidase Conjugate |
| TMB | tetramethylbenzidine |
| pfu | plaque-forming units |
| MDI | metered-dose inhaler |
| DPI | dry powder inhaler |

Definitions

As used above, and throughout the description of the invention, the following terms, unless otherwise indicated, shall be understood to have the following meanings.

“Compound of the invention”, and equivalent expressions, means the compound of formula (A), as hereinbefore described, which expression includes the pharmaceutically acceptable salt and the solvate, e.g., hydrate. similarly, reference to intermediates, whether or not they themselves are claimed, is meant to embrace the salts, and solvates, where the context so permits. For the sake of clarity, particular instances when the context so permits are sometimes indicated in the text, but these instances are purely illustrative and they are not intended to exclude other instances when the context so permits.

“Treating” and “treatment” means prevention, partial alleviation, or cure of the disease. The compound and compositions of this invention are useful in treating conditions that are characterized by the activation of NF-κB and/or enhanced levels of cytokines and mediators that are regulated by NF-κB including, but not limited to TNFα and IL-1β. Inhibition or suppression of NF-κB and/or NF-κB-regulated genes such as TNFα may occur locally, for example, within certain tissues of the subject, or more extensively throughout the subject being treated for such a disease. Inhibition or suppression of NF-κB and/or NF-κB-regulated genes such as TNFα may occur by one or more mechanisms, e.g., by inhibiting or suppressing any step of the pathway(s) such as inhibition of IKK. The term “NF-κB-associated condition ” refers to diseases that are characterized by activation of NF-κB in the cytoplasm (e.g., upon phosphorylation of IκB). The term“TNFα-associated condition ” is a condition characterized by enhanced levels of TNFα. In the instant specification, the term NF-κB-associated condition will include a TNFα-associated condition but is not limited thereto as NF-κB is involved in the activity and upregulation of other pro-inflammatory proteins and genes. The term “inflammatory or immune diseases or disorders” is used herein to encompass both NF-κB-associated conditions and TNFα-associated conditions, e.g., any condition, disease, or disorder that is associated with release of NF-κB and/or enhanced levels of TNFα, including conditions as described herein.

“Patient” includes both human and other mammals.

“Pharmaceutically effective amount” is meant to describe an amount of a compound, composition, medicament or other active ingredient effective in producing the desired therapeutic effect.

“substantially pure” is meant to refer to the compound where it is substantially free of biological or chemical constituents, e.g., isolated from a biological or chemical composition where biological or chemical components are co-isolated therewith, and wherein the analytical purity for the compound is preferably at least 70%. More preferred is where the analytical purity is at least 90%; even further preferred is where the analytical purity is at least 95%; also “substantially pure” is meant to refer to the compound where it is substantially free of prodrugs, e.g., Compound (B).

The invention also relates to a process for preparing the compound of the formula (A), as shown in the following Scheme.

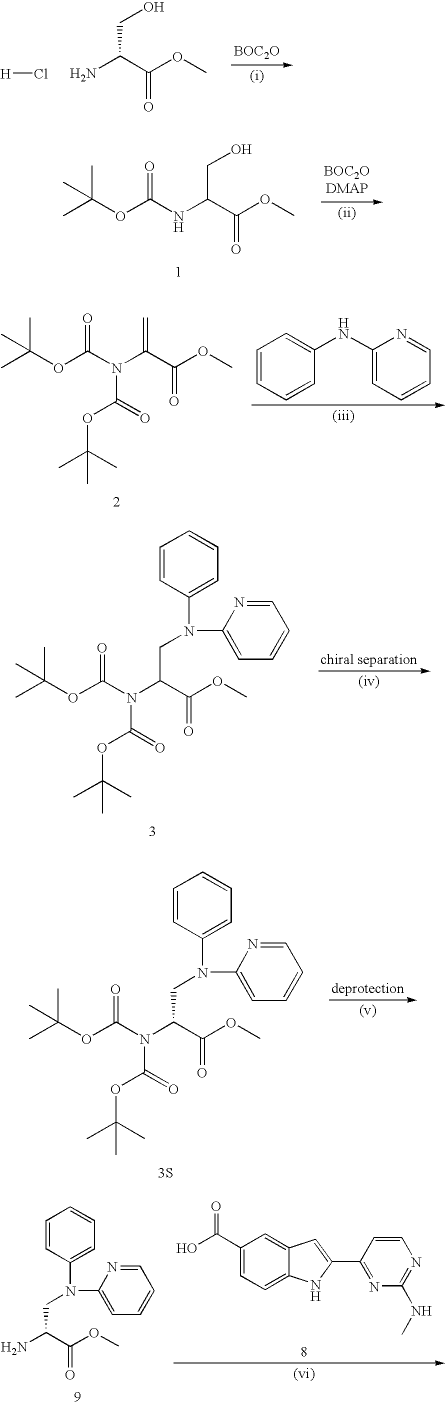

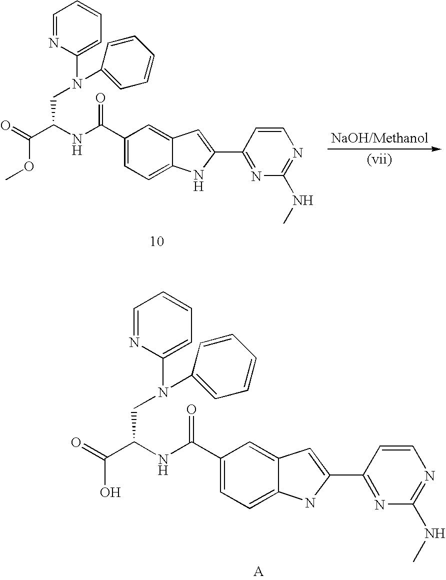

Starting compounds for the chemical reactions are known or they can be readily prepared using methods known from the literature. U.S. application Ser. No. 10/642,970 describes the preparation of the indole carboxylic acid intermediate (compound 8) used in the coupling step (vi) above. The compounds of the formulae (B), (C) and (D) are prepared as described in U.S. application Ser. No. 10/642,970, which is incorporated herein by reference.

Coupling methods of peptide chemistry that are well known to one skilled in the art (see, e.g., Houben-Weyl, Methoden der Organischen Chemie [Methods of Organic Chemistry], volumes 15/1 and 15/2, Georg Thieme Verlag, Stuttgart, 1974, the content of which is incorporated herein by reference), are advantageously used for condensing the compounds. Compounds such as carbonyldiimidazole, carbodiimides such as dicyclohexylcarbodiimide or diisopropylcarbodiimide (DIC), O-((cyano (ethoxycarbonyl)methylene)-amino-N,N,N′,N′-tetramethyluronium tetrafluoroborate (TOTU) or polyphosphoric acid (PPA) are suitable for use as condensing agents or coupling reagents.

The condensations can be carried out under standard conditions. In the condensation, it is necessary for the non-reacting amino groups that are present to be protected with reversible protecting groups. The same applies to carboxyl groups that are not involved in the reaction, with these groups preferably being present, during the condensation, as (C₁-C₆)-alkyl esters, benzyl esters or tert-butyl esters. An amino group protection is not necessary if the amino groups are still present in the form of precursors such as nitro groups or cyano groups and are only formed by hydrogenation after the condensation. After the condensation, the protecting groups that are present are eliminated in a suitable manner. For example, NO₂ groups (guanidino protection in amino acids), benzyloxycarbonyl groups and benzyl groups in benzyl esters can be eliminated by hydrogenation. The protecting groups of the tert-butyl type are eliminated under acidic conditions while the 9-fluorenylmethyloxy-carbonyl radical is removed using secondary amines.

The invention also relates to a pharmaceutical composition comprising a pharmaceutically effective amount of the compound of the formula (A) and a pharmaceutically acceptable carrier.

### EMBODIMENTS

Because of the pharmacological properties of the compound according to the invention, it is suitable for the treatment of all those patients suffering from or subject to conditions that can be ameliorated by the targeted administration of an inhibitor of IκB kinase to a site where the treatment is better effected by localized versus systemic activity, e.g., asthma, or chronic obstructive pulmonary disorder (COPD).

In practice, the compound of the present invention may be administered in pharmaceutically acceptable dosage form to humans and other animals by topical or systemic administration, including oral, inhalational, rectal, nasal, buccal, sublingual, vaginal, colonic, parenteral (including subcutaneous, intramuscular, intravenous, intradermal, intrathecal and epidural), intracisternal and intraperitoneal. It will be appreciated that the preferred route may vary with for example the condition of the recipient.

Intranasal, intratracheal, or inhalational administration, as well as aerosoliztion, are particular methods of administering the compound according to the invention.

Combination therapies may improve efficacy and decrease the risk of side effects compared with increasing the dose of a single agent. IKK inhibitors can be combined with bronchodilators including but not limited to short-acting β2-agonists; long-acting β2-agonists such as salmeterol and formoterol; anticholinergic agents such as ipratropium bromide and tiotropium bromide. IKK inhibitors can also be combined with methylxanthines such as theophylline.

Inhibitors of IKK2 can be combined with several anti-inflammatory therapies including but not limited to immunomodulators directed at various stages of the inflammatory cascade and directed to ameliorating inflammatory processes. Such therapies include, but are not limited to:

(A) Inhibitors of cellular recruitment and toxic inflammatory mediators including but not limited to phosphodiesterase-4 inhibitors; inhibitors of p38 mitogen-activated protein kinase; biopharmaceuticals such as anti-tumor necrosis factor-alpha, anti-interleukin-8, and anti-monocyte chemoattractant protein-1; inhibitors of adhesion molecules and chemotactic factors; and molecules that interfere with cell survival and clearance/apoptosis;

(B) Inhibitors of proteolytic enzymes including but not limited to inhibitors of neutrophil-derived serine proteases such as neutrophil elastase; and inhibitors of matrix metalloproteinases (MMPs) such as MMP-2, MMP-9 and MMP-12;

(C) Antioxidants including but not limited to N-acetylcysteine and inhibitors or scavengers of reactive oxygen species; and toxic peptides such as defensins that can directly cause cell injury;

(D) Inhibitors of mucus production including but not limited to inhibitors of mucous genes; and also mucus clearing agents such as expectorants, mucolytics, and mucokinetics; and

(E) Antibiotic therapy such as with a ketolide, for example Ketek®.

The drug combinations of the present invention can be provided to a cell or cells, or to a human patient, either in separate pharmaceutically acceptable formulations administered simultaneously or sequentially, formulations containing more than one therapeutic agent, or by an assortment of single agent and multiple agent formulations. However administered, these drug combinations form a pharmaceutically effective amount of components.

the treatment regimen/dosing schedule can be rationally modified over the course of therapy so that the lowest amounts of each of the pharmaceutically effective amount of compounds used in combination which together exhibit satisfactory pharmaceutical effectiveness are administered, and so the administration of such pharmaceutically effective amount of compounds in combination is continued only so long as is necessary to successfully treat the patient.

A pharmaceutical composition according to the invention is preferably produced and administered in dosage units, with each unit containing, as the active constituent, a particular dose of the compound. Pharmaceutically acceptable salts of the compound of formula (A) are within the scope of this invention. The term “salt(s)” means acid or base addition salts formed with acids and bases. In addition, the term “salt(s)” include zwitterion salts (inner salts), i.e., as the compound of formula (A) contains both a basic moiety, such as an amine or a pyridine or imidazole ring, and an acidic moiety, such as a carboxylic acid. Pharmaceutically acceptable (i.e., non-toxic, physiologically acceptable) salts are preferred, such as, for example, acceptable metal and amine salts in which the cation does not contribute significantly to the toxicity or biological activity of the salt. However, other salts may be useful, e.g., in isolation or purification steps that may be employed during preparation, and thus, are contemplated within the scope of the invention. Salts of the compounds of the formula (A) may be formed, for example, by reacting a compound of the formula (A) with an amount of acid or base, such as an equivalent amount, in a medium such as one in which the salt precipitates or in an aqueous medium followed by lyophilization.

Acid addition salts are formed with the compound of the invention bearing basic moiety(ies) such as an imino nitrogen, amino or mono or disubstituted group is present. Particular acid addition salts are the pharmaceutically acceptable acid addition salts, i.e., salts whose anion is non-toxic to a patient at a pharmaceutical dose of the salt, and so that the beneficial effects inherent in the free form of the compound are not vitiated by side effects ascribable to the anion. The salts chosen are chosen optimally to be compatible with the customary pharmaceutical vehicles and adapted for the form of applicable administration. Acid addition salts of the compound of this invention can be prepared by reaction of the free form of the molecule bearing the basic moiety with the appropriate acid, by the application or adaptation of known methods. For example, the acid addition salts of the compound of this invention can be prepared either by dissolving the free form of the molecule bearing the base moiety in water or aqueous alcohol solution or other suitable solvents containing the appropriate acid and isolating the salt by evaporating the solution, or by reacting the free form of the molecule bearing the base moiety and acid in an organic solvent, in which case the salt separates directly or can be obtained by concentration of the solution. Some suitable acids for use in the preparation of such salts are hydrochloric acid, hydrobromic acid, phosphoric acid, sulfuric acid, various organic carboxylic and sulfonic acids, such as acetic acid, citric acid, propionic acid, succinic acid, benzoic acid, tartaric acid, fumaric acid, mandelic acid, ascorbic acid, malic acid, methanesulfonic acid, toluenesulfonic acid, mandelic acid, ascorbic acid, malic acid, fatty acids, adipate alginate, ascorbate, aspartate, benzenesulfonate, benzoate, cyclopentanepropionate, digluconate, dodecylsulfate, bisulfate, butyrate, lactate, laurate, lauryl sulfate, maleate, hydroiodide, 2-hydroxy-ethanesulfonate, glycerophosphate, picrate, pivalate, palmoate, pectinate, persulfate, 3-phenylpropionate, thiocyanate, 2-naphtalenesulfonate, undecanoate, nicotinate, hemisulfate, heptanoate, hexanoate, camphorate, camphorsulfonate, and others.

The acid addition salts of the compound of this invention can also be used to regenerate the parent compound of the invention from the salts by the application of adaptation of known methods. For example, the parent compound of the invention can be regenerated from their acid addition salts by treatment with an alkali, e.g., aqueous sodium bicarbonate solution or aqueous ammonia solution.

Base addition salts are formed with the compound of the invention bearing the carboxy moiety. Particular base addition salts are the pharmaceutically acceptable base addition salts, i.e., salts whose cation is non-toxic to a patient at a pharmaceutical dose of the salt, so that the beneficial effects inherent in the free form of the compound are not vitiated by side effects ascribable to the anion. The salts chosen are chosen optimally to be compatible with the customary pharmaceutical vehicles and adapted for the form of applicable administration. Base addition salts of the compound of this invention can be prepared by reaction of the free form of the molecule bearing the acid moiety with the appropriate base, by the application or adaptation of known methods. For example, the base addition salts of the compound of this invention can be prepared either by dissolving the free form of the molecule bearing the acid moiety in water or aqueous alcohol solution or other suitable solvents containing the appropriate base and isolating the salt by evaporating the solution, or by reacting the free form of the molecule bearing the acid moiety and base in an organic solvent, in which case the salt separates directly or can be obtained by concentration of the solution. Some suitable bases for use in the preparation of such salts are those derived from alkali and alkaline earth metal salts or amines such as: sodium hydride, sodium hydroxide, potassium hydroxide, calcium hydroxide, aluminum hydroxide, lithium hydroxide, magnesium hydroxide, zinc hydroxide, ammonia, ethylenediamine, N-methyl-glucamine, lysine, arginine, ornithine, choline. N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, and the like.

The base addition salts of the compound of this invention can also be used to regenerate the parent compound of the invention from the salts by the application or adaptation of known methods. For example, the parent compound of the invention can be regenerated from their base addition salts by treatment with an acid, e.g., hydrochloric acid

In practice, the compound of the present invention is administered in a suitable formulation to patients such that its activity is particularly localized. It will be appreciated that the preferred route can be varied depending on the site of the condition for which administration is directed.

Pharmaceutically acceptable dosage forms refers to dosage forms of the compound of the invention, and includes, for example, powders, suspensions, sprays, inhalants, tablets, emulsions, and solutions, particularly suitable for inhalation. Techniques and formulations generally may be found in Remington's Pharmaceutical Sciences, Mack Publishing Co., Easton, Pa., latest edition.

If desired, and for more effective distribution, the compound can be microencapsulated in, or attached to, a slow release or targeted delivery systems such as biocompatible, biodegradable polymer matrices (e.g., poly(d,1-lactide co-glycolide)), liposomes, and microspheres and subcutaneously or intramuscularly injected by a technique called subcutaneous or intramuscular depot to provide continuous slow release of the compound(s) for a period of 2 weeks or longer.

The compound may also be sterilized, for example, by filtration through a bacteria retaining filter, or by incorporating sterilizing agents in the form of sterile solid compositions which can be dissolved in sterile water, or some other sterile medium immediately before use.

Formulations suitable for nasal or tracheal administration means formulations that are in a form suitable to be administered nasally or by inhalation to a patient. The formulation may contain a carrier, in a powder form, having a particle size for example in the range 1 to 500 microns (including particle sizes in a range between 20 and 500 microns in increments of 5 microns such as 30 microns, 35 microns, etc.). Suitable formulations wherein the carrier is a liquid, for administration as for example a nasal spray or as nasal drops, include aqueous or oily solutions of the active ingredient. Formulations suitable for aerosol administration may be prepared according to conventional methods and may be delivered with other therapeutic agents. MDI and DPI are feasible means for effecting inhalation therapy by administering a dosage form of the compound of the present invention.

Actual dosage levels of active ingredient(s) in the compositions of the invention may be varied so as to obtain an amount of active ingredient(s) that is (are) effective to obtain a desired therapeutic response for a particular composition and method of administration for a patient. A selected dosage level for any particular patient therefore depends upon a variety of factors including the desired therapeutic effect, on the route of administration, on the desired duration of treatment, the etiology and severity of the disease, the patient's condition, weight, sex, diet and age, the type and potency of each active ingredient, rates of absorption, metabolism and/or excretion and other factors.

Total daily dose of the compounds of this invention administered to a patient in single or divided doses to about 1000 mg, more particularly from about 50 mg to 300 mg, and, further particularly from about 10 mg to 100 mg. However, higher or lower daily doses can also be appropriate. The daily dose can be administered either by means of a once-only administration in the form of a single dosage unit, or of several smaller dosage units, or by means of the multiple administration of subdivided doses at predetermined intervals. The percentage of active ingredient in a composition may be varied, though it should constitute a proportion such that a suitable dosage shall be obtained. Obviously, several unit dosage forms can be administered at about the same time. A dosage may be administered as frequently as necessary in order to obtain the desired therapeutic effect. Some patients may respond rapidly to a higher or lower dose and may find much weaker maintenance doses adequate. For other patients, it may be necessary to have long-term treatments at the rate of 1 to 4 doses per day, in accordance with the physiological requirements of each particular patient. It goes without saying that, for other patients, it will be necessary to prescribe not more than one or two doses per day.

The formulations can be prepared in unit dosage form by any of the methods well known in the art of pharmacy. Such methods include the step of bringing into association the active ingredient with the carrier that constitutes one or more accessory ingredients. In general the formulations are prepared by uniformly and intimately bringing into association the active ingredient with liquid carriers or finely divided solid carriers or both, and then, if necessary, shaping the product.

Experimental

Mass-spectroscopic methods (FAB-MS, ESI-MS) are used for analyzing. Temperatures are given in degrees Celsius; RT denotes room temperature (from 22° C. to 26° C.). Abbreviations used are either explained or correspond to customary conventions to one skilled in the art.

The invention is exemplified through the following Examples.

### EXAMPLES

### Preparation 1

Synthesis of Methyl (3-(N-phenyl-N-2-pyridyl)amino)-2- (di-tert-butoxycarbonylamino)propionate (Scheme 1, Compound 3) 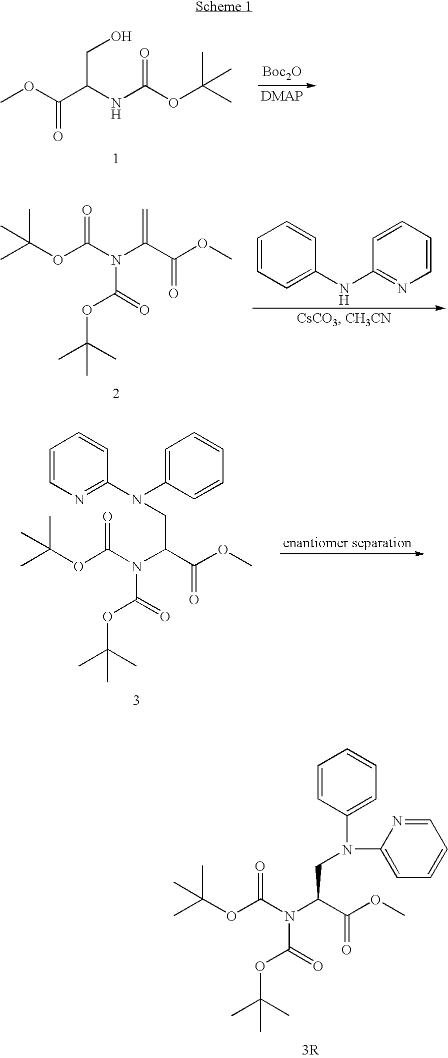
Methyl 2-(di-tert-butoxycarbonylamino(acrylate (Scheme 1, Compount 2) 50 g (0.228 mol) of tert-butoxycarbonyl)serine (1) were dissolved in 300 ml of acetonitrile. 107 g (0.493 mol) of di-tert-butyl dicarbonate and 2.64 g (22 mmol) of 4-(dimethylamino)pyridine (DMAP) were added. The mixture was stirred at room temperature overnight, after which the solvent was removed under reduced pressure and the residue was taken up in 500 mL of ethyl acetate. The organic phase was washed with 500 mL of 1 N HCl, dried using magnesium sulfate and the organic solvents were removed under reduced pressure. 23 g of acrylate 2 were obtained by crystallizing the residue from 200 mL of heptane at −30° C. and then filtering with suction. The mother liquor was concentrated and the residue was dissolved in 140 mL of acetonitrile. 31 g (0.142 mol) of di-tert-butyl dicarbonate and 1.26 g (10 mmol) of DMAP were added. After the mixture had been heated at 50° C. for 8 h, the solvent was removed in vacuo and the residue was taken up in 500 mL of ethyl acetate. The organic phase was washed with 400 mL of 1 N HCl and dried over magnesium sulfate. After the solvent had been removed in vacuo, a further 31.5 g of the acrylate 2 were obtained by crystallizing from heptane. Yield: 54.5 g (0.181 mol) 79%. Empirical formula C₁₄H₂₃NO_{6; M.W.=}301.34; MS ((2M*)+Na+) 625.7.¹H NMR (DMSO−d₆) 1.40 (s, 18 H), 3.74 (s, 3 H), 5.85 (s, 1 H), 6.28 (s, 1 H).
Methyl (3-(N-phenyl-N-2-pyridyl)amino)-2- (di-tert-butoxycarbonylamino)-propionate (Scheme 1, Compound 3)

4.96 g (16.5 mmol) of acrylate 2 were mixed with 5.6 g (33 mmol) of 2-anilinopyridine and 32.16 g (98.7 mmol) of cesium carbonate. 50 mL of acetonitrile were added and the mixture was stirred at 45° C. for 2 days. The solid was filtered off with suction through kieselguhr and washed 3 times with 100 mL portions of acetonitrile. The combined organic phases were evaporated and the residue was chromatographed on silica gel using 1:1 heptane/diethyl ether. 5.66 g (73%) of the ester 3 were obtained. Empirical formula C₂₅H₃₃N_{c}O₆; M.W.=4.71.56; MS (M+H( 472.2.

Separation of the Enantiomers (Scheme 1, Compound 3(S) and Compound 3(R))

Racemic amino ester 3 was prepared from the corresponding acrylic ester 2 and then resolved into enantiomers 3(S) and 3(R) by means of preparative HPLC using chiral stationary phases such as Chiralpak AD (Daicel) 100×380, RT, flow rate 300 mL/min. The purity of the enantiomers were determined by analytical HPLC such as Chiralpak-AD-H (Daicel) 4.6×250, 30°C., flow rate 1 ml/min, room temperature.

### Preparation 2

Synthesis of 2-(2-Methylaminopyrimidin-4-yl)-1H-indole-5-carboxylic acid (8) (Scheme 2, Compound 8) 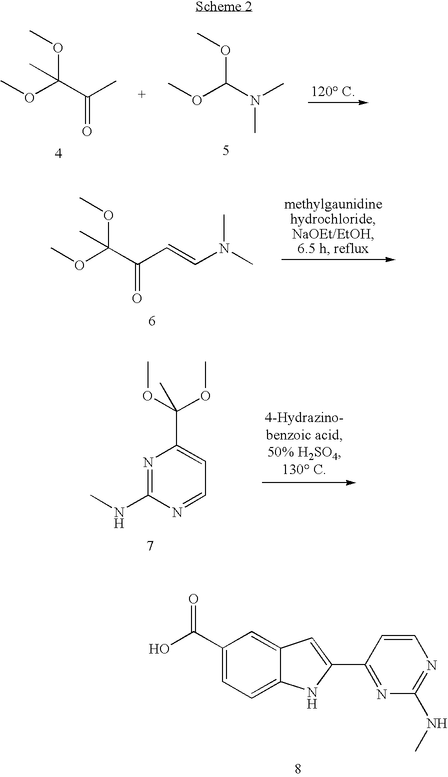
1-Dimethylamino-4,4-dimethoxypent-1-en-3-one (Scheme 2, Compound 6)

100 g (0.76 mol) of 3,3-dimethoxy-2-butanone (4) were stirred together with 90.2 g (0.76 mol) of N,N-dimethylformamide dimethyl acetal (5) at 120°( C. for 48 h. the methanol formed in the reaction was removed continuously from the reaction solution by means of distillation. Crystallization occurred spontaneously when the solution was cooled, with the crystallization being brought to completion by addition of a small amount of heptane. This resulted in 128.24 g of crude product 6 (yield 90%), which was reacted without any further purification. Empirical formula C₉H₁₇NO₃; M.W.=187.24; MS (M+H) 188.2. ¹H NMR (DMSO−d₆) 1.22 (s, 3 H), 2.80 (s, 3 H, 3.10 (s, 9 H), 5.39 (d,J=15 Hz, 1 H), 7.59 (d,J=14 Hz, 1 H).

[4-(1,1-Dimethoxyethyl)pyrimidin-2-yl]methylamine (Scheme 2, Compound 7)

1.22 g (53 mmol) of sodium was dissolved in 100 mL of absolute ethanol. 5.8 g (53 mmol) of methylguanidine hydrochloride and 10 g (53 mmol) of 1-dimethylamino-4,4-dimethoxypent-1-en-3-one (6) were added to this solution, while stirring, and the whole was heated at reflux for 4 h. To terminate the reaction, the ethanol was evaporated. The resulting product 7 was used for the subsequent reaction without any further purification. Yield: 11.5 g (58 mmol, quantitative): Empirical formula C₉H₂₅N₃O₂; M.W.=197.24; MS (M+H) 198.2. ¹H NMR (DMSO−d₆) 1.45 (s, 3 H), 2.78 (s, 3 H), 3.10 (s, 6 H), 6.75 (d,J=3 Hz, 1 H), 7.0-7.1 (s(b), 1 H), 8.30 (d,J=3 Hz, 1 H).

2-(2-Methylaminopyrimidin-4-yl)-1H-indole-5-carboxylic acid (Scheme 2, Compound 8)

5 g (25 mmol) of [4-(1,1-dimethoxyethyl)pyrimidin-2-yl]methylamine (7) and 3.85 g of 4-hydrazinobenzoic acid were added, at room temperature and while stirring, to 150 mL of 50% sulfuric acid, and the mixture was heated at 130° C. for 4 h. The methanol that was formed in the reaction was removed continuously from the reaction solution by means of distillation. After it had been cooled down to 10°C., the reaction mixture was poured onto 200 mL of ice and adjusted to a pH of about 5.5 with concentrated sodium hydroxide solution. The precipitate of sodium sulfate and product mixture formed was filtered off and the filter residue was extracted several times with methanol. The combined methanol extracts were concentrated and the product 8 was purified by means of flash chromatography (CH₂Cl₂/methanol 9:1). Yield: 0.76 g (11%); Empirical formula C₁₄H₁₂N₄O₂; M.W.=268.28; MS (M+H) 269.1. ¹H NMR (DMSO−d₆) 2.95 (s, 31 H), 6.90 7.10 (s(b), 1 H), 7.18 (d,J=3 Hz, 1 H), 7.4 (s, 1 H), 7.58 (d,J=4.5 Hz, 1 H), 7.80 (d,J=4.5 Hz, 1 H), 8.30 (s, 1 H), 8.38 (d,J=3 Hz, 1 H), 11.85 (s, 1 H), 12.40-12.60 (s(b), 1 H).

### Example 1

Synthesis of 2-{[2-(2-Methylamino-pyrimidin-4-yl)-1H-indole-5-carbonyl]-amino}-3-(phenyl-pyridin-2-yl-amino)-propionic acid (A) 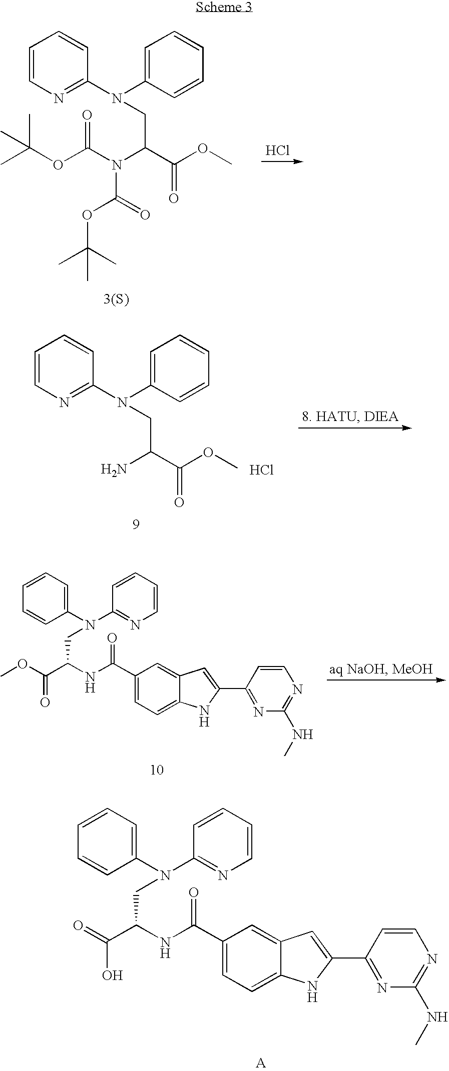
2-{[2-Methylamino-pyrimidin-4-yl)-1H-indole-5-carbonyl]-amino}-3-(phenyl-pyridin-2-yl-amino)-propionic acid, methyl ester (Scheme 3, Compound 10)

2.9 g of the S enantiomer of 3 (3 (S)) were dissolved in 30 mL of dioxane and the solution was cooled down to 0° C. 30 mL of 4 N HCl in dioxane were added, after which the mixture was allowed to come to room temperature and was then stirred for 12 h. The solvent was removed in vacuo. The residue was taken up in 30 mL of DMD (solution A). 2.47 g (9.2 mmol) of the acid 8 were dissolved in 30 mL of DMF and cooled down to 0° C. 4.21 g of HATU and 6.4 mL of DIEA were added. After the mixture had been stirred at 0° C. for 45 minutes, it was allowed to come to RT and solution A was added. The mixture was stirred at RT for 12 h. The solvent was removed in vacuo and the residue was partitioned between 300 mL of a saturated solution of NaHCO₃ and 300 mL of ethyl acetate. The aqueous phase was extracted 3 times with 100 mL portions of ethyl acetate and the combined organic phases were washed with 400 mL of a saturated solution of NaCl. The organic phase was dried with magnesium sulfate. The solvents were removed under reduced pressure and the residue was chromatographed on silica gel using 1:3 heptane/ehtyl acetate. 1.78 g (55%) of the ester 10 was obtained. Empirical formula C₂₉H₂₇N₇O₃; M.W.=521.58; MS (M+H) 522.2.

2-{[2-(2-Methylamino-pyrimidin-4-yl)-1H-indole-5-carbonyl]-amino}-3-(phenyl-pyridin-2-yl-amino)-propionic acid (Scheme 3,Compound A)

2.0 g (3.8 mmol) of the methyl ester 10 were dissolved in 200 mL of methanol. 1 mL of 2 N aqueous NaOH was added and the mixture was stirred at room temperature for 12 h. After the solvents had been evaporated, the residue was dissolved in water and the pH was adjusted to ˜5 using a saturated solution of NaH₂PO₄. the resulting precipitate was filtered off and washed with water. After drying under reduced pressure of about 1 mbar at 40°C., 1.95 g (quantitative yield) of the acid A was isolated. Empirical formula C₂₈H₂₅N₇O₃; M.W.=507.56;MS (M+H)508.3. ¹H NMR (DMSO−d₆) 2.95 (s, 3 H), 4.22-4.50 (m, 2 H), 4.65-4.72 (m, 1 H), 6.29-6.36 (d, 1 H), 6.70-6.79 (m, 1 H), 6.90- 7.10 (sb, 1 H), 7.13-7.19 (m, 1 H), 7.22-7.38 (m, 5 H), 7.40-7.48 (m, 3 H), 7.50-7.55 (m, 1 H), 7.57-7.60 (m, 1 H), 7.96 (bs, 1 H), 8.34-8.40(m, 2 H), 8.80-8.90 (d, 1 H), 11.80 (s, 1 H).

In Vitro Test Procedure

IKK-Enzyme ELISA

The assay buffer has the following composition (50 mM HEPES, 10 mM MgCl2, 10 mM β-Glycerophosphate, 2 μM Microcystin-LR, 0.01% NP-40, 5 mM DTT).

The IKK enzyme preparation was diluted 1:50 (in-house-made preparation) plus test compound in DMSO (final concentration in well: 2%).

The assay procedures were as follows:

Incubation of enzyme and compound for 30 min;

Addition of 1 mM ATP or 50 μM ATP;

pSer36-IkB Peptide (Substrate): 40 μM;

Incubation for 45 min and Addition of anti-pSer32-pSer36-IkB Peptid-antibody;

Incubation for 45 min and transfer to protein-G-coated plate;

Incubation for 90 min followed by 3× washing;

Addition of streptavidin-HRP, then incubation 45 min followed by 6× washing;

Addition of TMB and Incubation for 15 min; and

Stop solution and read using photometer.

The results from the in vitro profiling are shown in Table I below.

TABLE I

| | IC₅₀ (nM) | IC₅₀ (nM) |
|---|---|---|
| Compound | 1 mM ATP | 50 μM ATP |
| A | 0.08 | 0.4 |
| B | 56.4 | 0.8 |
| C | 378 | 16.8 |
| D | 3.8 | — |

In the IKK-enzyme ELISA described above, at a 1 mM ATP, the compound of formula (A) exhibits a 705, 4,725 and 47.5 times greater IκB kinase IC₅₀ than Compounds (B), (C) and (D), respectively. This data demonstrates an unexpectedly significantly superior activity for Compound (A) relative to Compounds (B), (C) and (D).

In Vivo Test Procedures Comparison Between Compounds A and B

NF-κB-induced gene expression contributes significantly to the pathogenesis of inflammatory diseases such as asthma and arthritis. IκB kinase (IKK) is the converging point for the activation of NF-κB by a broad spectrum of inflammatory agonists.

IKK is a multisubunit complex that contains two catalytic subunits, IKK-1 (also known as IKK-α) and IKK-2 (also known as IKK-β), and the regulatory subunit IKK-γ. Gene knock out studies have clearly demonstrated that IKK-2 or IKK-β subunits of the IKK complex are required for NF-κB activation by all known pro-inflammatory stimuli including lipopolysaccharide (LPS), and IL-1β. Accordingly, IKK-β-deficient cells are defective in activation of IKK and NF-κB in response to either tumor necrosis factor alpha (TNFα) or interleukin-1β (IL-1β).

In house bio-imaging data have also shown that IL-1β-induced NF-κB activity in the lung is inhibited by the administration of a dominant negative form of IKKβ (Adv-IKK-2 DN). Thus a selective inhibitor of IKK-β would not only be of great interest as a potential anti-inflammatory agent but also as a valuable tool to understand the mechanisms regulating NF-κB activation by these inflammatory agonists.

A. NF-κB-Luciferase Reporter Mouse Model

Methods employed for mouse imaging studies with Compound (A) and Compound (B).

General Description

Compounds were used as nanomilled suspensions in 0.2% Tween-80 in PBS and were dosed through the intranasal route.

Intranasal drug and inflammatory stimulus administration: Mice are anesthetized in 4% is isoflurane gas in oxygen. A volume of 25 μl is applied to each nostril and the mice are allowed to breathe in the suspension.

Balb/c female mice at 6-8 weeks of age were used for studies in which AdV-NFκB-luciferase reporter was instilled in the lung. To imagine mice, they are anesthetized with 4% isoflurane in oxygen. Luciferin is delivered i.p. at a dose of 150 mg/kg. 10 minutes after luciferin injection, the animals are imaged in an IVIS200 system (Xenogen) with a one-minute bioluminescent exposure. Alternatively, 10-15 minutes post luciferin administration, mice are rapidly euthanized and internal tissues are dissected and imaged ex vivo.

B. Dose Response Determination

1-2×10⁸ pfu adenovirus-NFκB-luciferase is delivered intranasally 3-5 days prior to stimulation (i.n.) with an inflammatory stimulus (IL-1β or LPS). Animals are dosed i.n. with 0.3-10 mg/kg of compound 30 min—1 h prior to challenge with 0.5 μg LPS or 50 ng IL-1β. Animals are then imaged once or several times from 1 h to 24 h after the inflammatory stimulus is administered.

The results from the in vivo procedures are as follows.

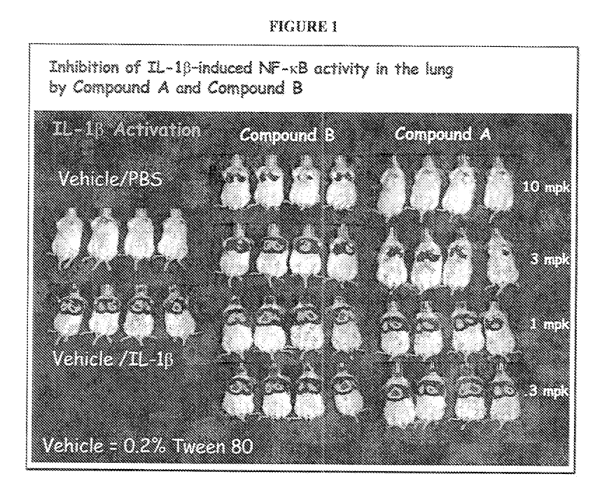

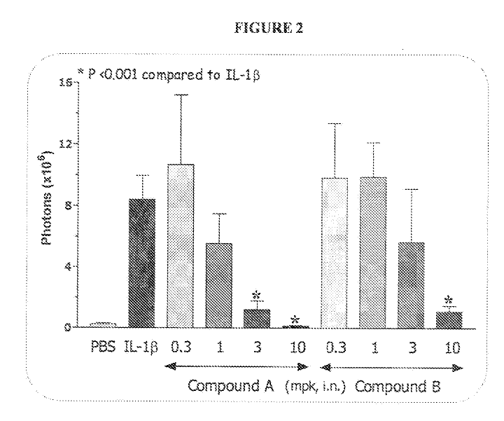

The effects of Compound (A) and Compound (B) on IL-1β-induced NF-κB activation are shown in FIGS. 1 and 2. While both compound inhibited in a dose-dependent manner NF-κB activity, Compound (A) showed superior efficacy with an estimated ED₅₀ around 1 mg/kg.

C. Pharmacokinetic Procedures

Male Hartley guinea pigs (450-550 g) previously sensitized with ovalbumin were used for the determination of compound levels in lung and plasma. Nanomilled suspensions of Compound (A) and Compound (B) were dosed via intra-tracheal instillation at 0.01, 0.03, 0.1 and 0.3 mg/kg. One hour after dosing, animals were euthanized (Euthasol), and 1 mL blood samples were obtained by cardiac puncture and collected into heparin-coated syringes. Plasma was separated from the cellular component of the blood by centrifugation, and stored at −80° C. until assayed. Lungs were dissected out, blotted dry, weighted and stored in 20-25 mL glass vials individually at −80° C. until assayed for compound levels.

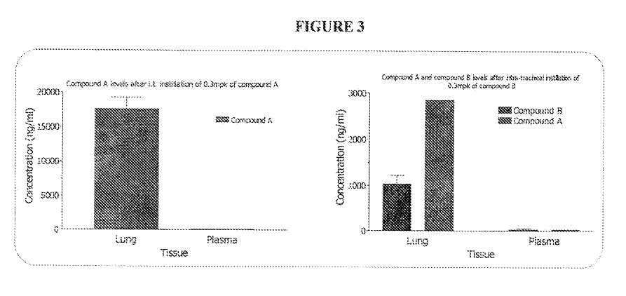

Key differences between Compound (A) and Compound (B) pharmacokinetic profiles are illustrated in FIG. 3 using the finding from the highest dose group (0.3 mg/kg).

FIG. 3 shows that lung exposure to either Compound (A) or Compound (B) after i.t. instillation of Compound (B) is low relative to Compound (A) after i.t. administration, suggesting that Compound (B) is rapidly absorbed from the lung.

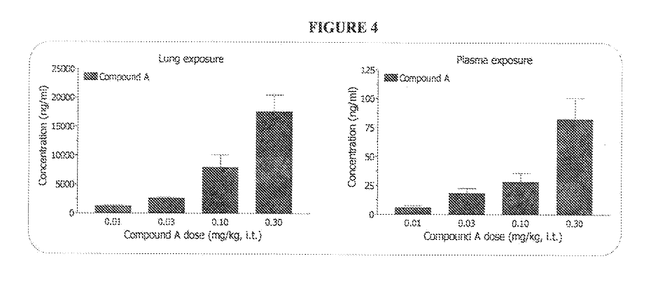

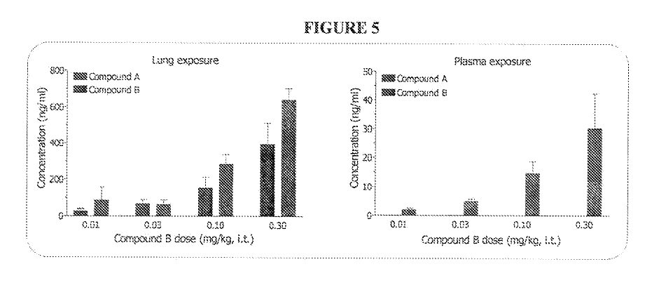

FIGS. 3 to 5 also show that Compound (B) would be a weaker candidate for inhalation because 1) It is highly systemically distributed upon exposure, when administered intratracheally; 2) Compound (B) is a prodrug of Compound (A) having a different exposure profile; and 3) Compound (B) produces reduced exposure to Compound (A) in the lung than is obtained by dosing directly with Compound (A).

Compound (A) is a stronger inhalation candidate than Compound (B) because 1) Compound (A) has low systemic exposure after i.t. and PO administration; and 2) Compound (A) should have longer lung residency time.

In addition, support is found that Compound (B) is highly systemically available when administered orally.

The lung to plasma ratio for Compound (A) ranges from 143 to 284 (depending on the dose) whereas the lung to plasma ratio for Compound (B) ranges from 13 to 44 (depending on the dose). These ratios were obtained by dividing the lung compound levels to that of the corresponding plasma levels at the same dose.

We claim:

1. A substantially pure compound of formula (A) 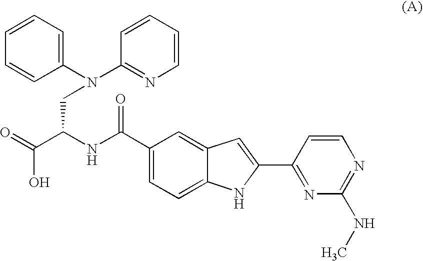
or a pharmaceutically acceptable salt or solvate thereof.

2. A pharmaceutical composition comprising a pharmaceutically effective amount of a compound of formula (A) and a pharmaceutically acceptable carrier.

3. A method for treating a patient suffering from, or subject to, a pathological condition that can be ameliorated by inhibiting IKK-2 comprising administering to said patient a pharmaceutically effective amount of the compound according to claim 1

4. The method according to claim 3 wherein the administering is carried out to result in localized activity.

5. The method according to claim 3 wherein the pathological condition is asthma, rhinitis, chronic obstructive pulmonary disorder or chronic obstructive pulmonary disorder exacerbations.

6. The method according to claim 3 wherein the administering is intratracheal, intranasal, inhalational, or by acrosolization administration.

7. A method for treating a patient suffering from asthma, comprising administering to the patient a pharmaceutically effective amount of a compound of claim 1.

8. A method for treating a patient suffering from rhinitis, comprising administering to the patient a pharmaceutically effective amount of a compound of claim 1.

9. A method for treating a patient suffering from chronic obstructive pulmonary disorder, comprising administering to the patient a pharmaceutically effective amount of a compound of claim 1.

10. A method for treating a patient suffering from chronic obstructive pulmonary exacerbations disorder, comprising administering to the patient a pharmaceutically effective amount of a compound of claim 1.

11. The pharmaceutical composition according to claim 2 further comprising a pharmaceutically effective amount of a compound selected from the group consisting of a bronchodilator, a long-acting beta-2 agonist, an anticholinergic agents, a methylxanthine and an anti-inflammatory therapy, in admixture with a pharmaceutically acceptable carrier.

12. The pharmaceutical composition according to claim 11, wherein the bronchodilator is a short-acting beta 2-agonist; the long-acting beta 2-agonist is selected from salmeterol and formoterol; the anticholinergic agent is selected from ipratropium bromide and tiotropium bromide; the methylxanthine is theophylline; and the anti-inflammatory therapy is selected from inhibitors of cellular recutiment and toxic inflammatory mediators, inhibitors of proteolytic enzymes, antioxidants, inhibitors of mucus production and antibiotic therapy.

13. A method for treating according to claim 3 further comprising administering a pharmaceutically effective amount of a compound selected from the group consisting of a bronchodilator, a long-acting beta-2 agonist, and anticholinergic agents, a methylxanthine and an anti-inflammatory therapy, in admixture with a pharmaceutically acceptable carrier.

14. A method for treating according to claim 13 wherein the bronchodilator is a short-acting beta 2-agonist; the long-acting beta 2-agonist is selected from salmeterol and formoterol; the anticholinergic agent is selected from ipratropium bromide and tiotropium bromide; the methylxanthine is theophylline; and the anti-inflammatory therapy is selected from inhibitors of cellular recutiment and toxic inflammatory mediators, inhibitors of proteolytic enzymes, antioxidants, inhibitors of mucus production and antibiotic therapy.
